(19) United States
(12) Patent Application Publication
(10) Pub. No.: US 2022/0105054 A1
(43) Pub. Date: Apr. 7, 2022

(54) ABSORPTION ENHANCER OF CINNAMIC ACID DERIVATIVE

(71) Applicant: YAMADA BEE COMPANY, INC., Tomata-gun (JP)

(72) Inventors: Masayuki YAMAGA, Tomata-gun (JP); Hiroko TANI, Tomata-gun (JP)

(21) Appl. No.: 17/487,430

(22) Filed: Sep. 28, 2021

(30) Foreign Application Priority Data
Oct. 2, 2020 (JP) 2020-167641

(51) Int. Cl. A61K 31/12; A61K 31/192; A61K 9/08

(52) U.S. Cl. CPC A61K 31/12; A61K 9/08; A61K 31/192

(57) ABSTRACT

The present invention provides an absorption enhancer of a cinnamic acid derivative containing curcumin as an active ingredient, the cinnamic acid derivative is at least one selected from the group consisting of a compound represented by formula (1):

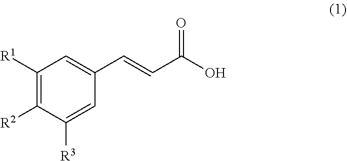

wherein R¹ represents a hydrogen atom, a hydroxyl group, a dimethylallyl group, 3-formyl-2-butenyl group, or (E)-3-methyl-4-hydroxy-2-butenyl group; R² represents a hydrogen atom, a hydroxyl group, or a dihydrocinnamoyloxy group; and R³ represents a hydrogen atom, a hydroxyl group, or a dimethylallyl group, or formula (2):

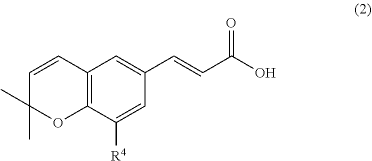

wherein R⁴ represents a hydrogen atom or a dimethylallyl group.

### CROSS-REFERENCE TO RELATED APPLICATION

This application claims the benefit of Japanese patent application No. 2020-167641 filed on Oct. 2, 2020, the disclosure of which is herein incorporated by reference in its entirety.

### TECHNICAL FIELD

The present invention relates to an absorption enhancer of a cinnamic acid derivative.

### BACKGROUND

Cinnamic acid derivatives such as artepillin C are known to have various physiological activities and blended into food such as supplements (Paulino, et al., Anti-inflammatory effects of a bioavailable compound, Artepillin C, in Brazilian propolis, European Journal of Pharmacology, 587 (2008) 296-301).

### SUMMARY

For supplements, a specific intake is recommended to effectively exhibit the physiological activity of active ingredients contained. However, in order for consumers to continue ingesting supplements on a daily basis, the lower required intake of supplements is preferred. Therefore, it is desirable to be able to absorb the cinnamic acid derivatives into the body more efficiently even when the intake thereof is small.

An object of the present invention is to provide an agent capable of enhancing absorption of a cinnamic acid derivative having a specific structure into the body.

The present invention provides an absorption enhancer of a cinnamic acid derivative, comprising curcumin as an active ingredient, wherein the cinnamic acid derivative is at least one selected from the group consisting of a compound represented by formula (1):

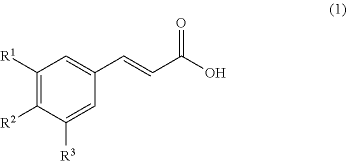

wherein R¹ represents a hydrogen atom, a hydroxyl group, a dimethylallyl group, 3-formyl-2-butenyl group, or (E)-3-methyl-4-hydroxy-2-butenyl group; R² represents a hydrogen atom, a hydroxyl group, or a dihydrocinnamoyloxy group; and R³ represents a hydrogen atom, a hydroxyl group, or a dimethylallyl group, or formula (2):

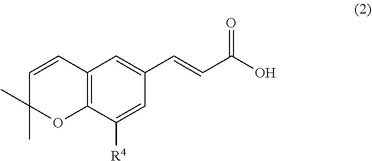

wherein R⁴ represents a hydrogen atom or a dimethylallyl group.

The cinnamic acid derivative may be derived from propolis.

The cinnamic acid derivative may be at least one selected from the group consisting of artepillin C, drupanin, p-coumaric acid, culifolin, capillartemisin A, 3,4-dihydroxy-5-prenyl-(E)-cinnamic acid, and 2,2-dimethylchromene-6-(E)-propenoic acid.

The present invention also provides a method for enhancing absorption of the cinnamic acid derivative, comprising administrating an effective amount of curcumin to a subject in need thereof, wherein the cinnamic acid derivative is at least one selected from the group consisting of a compound represented by formula (1) or (2).

An agent capable of enhancing absorption of a cinnamic acid derivative having a specific structure into the body is provided by the present invention.

### DETAILED DESCRIPTION

Hereinafter, suitable embodiments of the present invention will be described in detail. However, the present invention is not limited to the following embodiments.

An absorption enhancer of a cinnamic acid derivative according to the present embodiments contains curcumin as an active ingredient. The cinnamic acid derivative is at least one selected from the group consisting of a compound represented by formula (1):

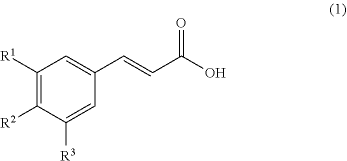

wherein R¹ represents a hydrogen atom, a hydroxyl group, a dimethylallyl group, 3-formyl-2-butenyl group, or (E)-3-methyl-4-hydroxy-2-butenyl group; R² represents a hydrogen atom, a hydroxyl group, or a dihydrocinnamoyloxy group; and R³ represents a hydrogen atom, a hydroxyl group, or a dimethylallyl group, or formula (2):

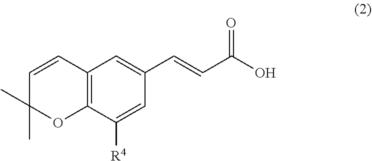

wherein R⁴ represents a hydrogen atom or a dimethylallyl group.

Curcumin is a pigment ingredient contained in a plant such as turmeric (Curcuma longa). Curcumin can be extracted from, for example, C. longa rhizome by known methods. The C. longa extracts may be obtained from C. longa rhizome by using water, hot water, an organic solvent such as ethanol, or a mixed liquid thereof as extraction solvents. Examples of the absorption enhancer according to the present embodiments may contain the C. longa rhizome or an extract thereof as a curcumin source. The absorption enhancer may contain a C. longa-derived ingredient other than curcumin Curcumin may have improved absorbability by known methods such as atomization. Curcumin may also be synthesized by known methods.

The content of curcumin in the absorption enhancer according to the present embodiment may be, for example, 1% by mass or more, 3% by mass or more, 5% by mass or more, 10% by mass or more, 15% by mass or more, or 17% by mass or more, and may be 40% by mass or less, 30% by mass or less, 25% by mass or less, or 23% by mass or less as a solid content relative to the total amount of the absorption enhancer.

The absorption enhancer according to the present embodiment can be used as a solid content of curcumin for an adult with a weight of 60 kg, for example, in a dose of 1 mg to 1000 mg per day; and it is preferable to use the absorption enhancer in a dose of 10 to 500 mg and it is more preferable to use the absorption enhancer in a dose of 100 to 350 mg.

Containing curcumin as an active ingredient, the absorption enhancer according to the present embodiment can enhance the absorption of the cinnamic acid derivative represented by formula (1) or (2) (hereinafter, simply referred to as a “cinnamic acid derivative”) into the body.

Specific examples of the cinnamic acid derivatives include artepillin C, drupanin, p-coumaric acid, culifolin, capillartemisin A, 3,4-dihydroxy-5-prenyl-(E)-cinnamic acid, 2,2-dimethylchromene-6-(E)-propenoic acid, cinnamic acid, p-coumaric acid, caffeic acid, and drupanal. The cinnamic acid derivatives may be synthetic products or may be derived from natural products, such as propolis. Substituents in formula (1) or (2) for each compound are shown in Table 1.

TABLE 1
Formula (1)

| | R₁ | R₂ | R₃ |
|---|---|---|---|
| Cinnamic acid | H | H | H |
| p-Coumaric acid | H | OH | H |
| Caffeic acid | OH | OH | H |
| Drupanin | CH₂CH═C(CH₃)₂ | OH | H |
| 3,4-dihydroxy-5-prenyl-(E)- | CH₂CH═C(CH₃)₂ | OH | OH |
| cinnamic acid | | | |
| Drupanal | CH₂CH═C(CH₂)CHO | OH | H |
| Artepillin C | CH₂CH═C(CH₃)₂ | OH | CH₂CH═C(CH₃)₂ |
| Capillartemisin A | CH₂CH═C(CH₂)CH₂OH | OH | CH₂CH═C(CH₃)₂ |
| Baccharin | CH₂CH═C(CH₃)₂ | OCO(CH₂)₂Ph | H |

Formula (2)

| | R4 | | |
|---|---|---|---|
| 2,2-dimethylchromene-6-(E)- | H | — | — |
| propenoic acid | | | |
| Culifolin | CH₂CH-C(Me)₂ | — | — |

It is preferable that the cinnamic acid derivative be at least one selected from the group consisting of artepillin C, drupanin, p-coumaric acid, culifolin, capillartemisin A, 3,4-dihydroxy-5-prenyl-(E)-cinnamic acid, and 2,2-dimethylchromene-6-(E)-propenoic acid. The absorption enhancer according to the present embodiment has an absorption enhancing effect, especially on these cinnamic acid derivatives.

The absorption enhancer may be ingested with the cinnamic acid derivative, or the cinnamic acid derivative may be ingested within a certain time, for example, within an hour or within 30 minutes, after ingesting the absorption enhancer.

The absorption enhancer according to the present embodiment may further contain at least one selected from the group consisting of the cinnamic acid derivatives represented by formula (1) or (2) in addition to curcumin as an active ingredient. It is preferable that the absorption enhancer contain both curcumin and the cinnamic acid derivative since it is easy to ingest curcumin and the cinnamic acid derivative at the same time and in the appropriate ratio.

When the absorption enhancer contains the cinnamic acid derivative, the amount of each cinnamic acid derivative represented by formula (1) or (2) contained in the absorption enhancer or the total amount thereof may be, for example, 0.001% by mass or more, 0.01% by mass or more, 0.1% by mass or more, 1% by mass or more, 3% by mass or more, 4% by mass or more, or 5% by mass or more, and may be 10% by mass or less, 5% by mass or less, 3% by mass or less, 1% by mass or less, 0.1% by mass or less, or 0.01% by mass or less as a solid content relative to the total amount of the absorption enhancer.

A cinnamic acid derivative source contained in the absorption enhancer may be, for example, propolis. When the absorption enhancer contains curcumin and propolis, the content ratio of curcumin to propolis contained in the absorption enhancer may be, for example, 1 to 10:1, 2 to 8:1, 2 to 6:1, or 3 to 5:1.

When the absorption enhancer contains curcumin and propolis, the amount of propolis contained in the absorption enhancer may be, for example, 1% by mass or more, 3% by mass or more, 4% by mass or more, 5% by mass or more, 7% by mass or more, 10% by mass or more, or 15% by mass or more, and may be 70% by mass or less, 50% by mass or less, 30% by mass or less, 20% by mass or less, 10% by mass or less, 8% by mass or less, or 5% by mass or less as a solid content relative to the total amount of the absorption enhancer.

Propolis capable of being used in the absorption enhancer can be obtained, for example, as an apicultural product according to a conventional method. The propolis may be derived from any plant, such as alecrim, eucalyptus, poplar, and Clusia spp. Alecrim is Baccharis dracunculifolia of the genus Baccharis of the family Asteraceae.

Propolis may be made, for example, in Japan, Brazil, China, European countries, Oceania, the United States of America, Argentina, Uruguay, Paraguay, Russia, Hawaii, Taiwan, Australia, New Zealand, Turkey, and Indonesia. Brazilian propolis is mainly derived from alecrim. Brazilian propolis is characterized by high cinnamic acid derivative content. It is preferable that the absorption enhancer according to the present embodiment contain the Brazilian propolis.

Propolis may be of any rank, such as brown, red, yellow, green, super green, and ultra-green, and among them, green, super green, or ultra-green ranked propolis is preferred. These ranks are determined by the artepillin C content in the propolis. The propolis with the content of artepillin C of 3% by mass or more is referred to as green propolis.

It is preferable that the propolis be derived from bees belonging to the genus Apis of the family Apidae, and among the genus Apis, derived from Apis mellifera. A. mellifera is considered to comprise 24 to 28 subspecies, and propolis from any of these subspecies may be used. In particular, it is preferable to use propolis derived from the Africanized honey bee, a hybrid of African honey bee (A. mellifera scutellata), a subspecies of A. mellifera, with other European subspecies of A. mellifera.

Propolis may be, for example, a propolis original lump, or may be processed propolis obtained by subjecting the propolis original lump to some sort of processing. The processed propolis may be obtained by subjecting the propolis original lump to processing, such as grinding, extraction, concentration or pulverization of an extract, and granulation of powder, or may be an extraction residue remaining after extraction. In other words, the processed propolis may be, for example, a ground product of propolis, an extract, a concentration extract, extract powder, an extract granule, and an extraction residue. The extraction may be, for example, water extraction, hydrophilic organic solvent extraction, or supercritical extraction. Examples of hydrophilic organic solvents include ethanol, glycerin, and 1,3-butylene glycol. The propolis extract may be obtained by extraction from the propolis original lump or by further extraction from the residue after the extraction. The processing method may be one or a combination of two or more. The hydrophilic organic solvent extract of propolis is preferable as processed propolis since the active ingredient of propolis is efficiently extracted with good balance in a short time. It is preferable that the processed propolis be an ethanol extract of propolis.

Commercially available products may be used as propolis. Examples of the commercially available products containing propolis include Propolis 300, Propolis Liquid 30 (made in Brazil), Propolis Grain, Propolis Granular APC, Propolis Mild, and Propolis Drink manufactured by Yamada Bee Company, Inc.; Neo Propolis Grain, Propolis Grain, Propolis Liquid, Propolis Mild Liquid, and Eucalypolis manufactured by Morikawa Kenkodo Kk; and L'abeille Propolis (liquid type), L'abeille Propolis (capsule type), and L'abeille Propolis honey manufactured by L'abeille Co., Ltd.

The absorption enhancer according to the present embodiment is suitable for oral administration. The absorption enhancer may be administered once a day or may be administered more than once, such as twice a day and three times a day.

An administration subject of the absorption enhancer according to the present embodiment may be a person who ingests, for example, a cinnamic acid derivative represented by formula (1) or (2), or a composition or food containing the cinnamic acid derivative (for example, propolis) to enjoy physiological activities thereof. It is possible to efficiently absorb the cinnamic acid derivative into the body by ingesting the absorption enhancer according to the present embodiment even when the intake of the cinnamic acid derivative is small, thereby reducing the intake of the cinnamic acid derivative required to obtain desired physiological activities. It will be easier to continuously ingest the cinnamic acid derivative on a daily basis by reducing the required intake thereof.

The absorption enhancer can be used as pharmaceuticals, quasi-drugs, or food compositions themselves, and can also be used as ingredients in pharmaceuticals, quasi-drugs, or food compositions.

In addition to curcumin and the cinnamic acid derivative, the absorption enhancer according to the present embodiment may further contain other physiologically active ingredients. Examples of the other physiologically active ingredients include a ginkgo leaf extract, phosphatidylserine, a coffee fruit extract, and a Gotu Kola extract. The absorption enhancer may contain one of the other physiologically active ingredients or a combination of two or more thereof. The amount of each of these physiologically active ingredients or the total amount thereof may be, for example, 1% by mass or more, 3% by mass or more, 5% by mass or more, 8% by mass or more, or 10% by mass or more, and may be 60% by mass or less, 50% by mass or less, 40% by mass or less, 30% by mass or less, 25% by mass or less, 20% by mass or less, 15% by mass or less, 10% by mass or less, 5% by mass or less, or 3% by mass or less in terms of solid content relative to the total amount of the absorption enhancer.

The absorption enhancer according to the present embodiment may further contain, for example, pharmaceutically acceptable ingredients (for example, excipients, binding materials, lubricants, disintegrators, emulsifiers, surfactants, base agents, solubilizing agents, and suspending agents), and ingredients acceptable as food (for example, minerals, vitamins, flavonoids, quinones, polyphenols, amino acids, nucleic acids, essential fatty acids, refrigerants, binders, sweeteners, disintegrators, lubricants, colorants, flavoring agents, stabilizers, preservatives, controlled-release agents, surfactants, solubilizers, and wetting agents).

The absorption enhancer according to the present embodiment may have any forms such as solids, liquids, and pastes, and may have any dosage forms such as tablets (including uncoated tablets, sugar-coated tablets, effervescent tablets, film-coated tablets, chewable tablets, and troches), capsules, pills, powders (powdered drugs), fine granules, granules, liquids agents, suspensions, emulsions, syrups, pastes, and injections (including those blended with distilled water or infusions such as amino acid infusions or electrolyte infusions to prepare a liquid agent at the time of use). These various formulations can be prepared, for example, by mixing an absorption enhancer obtained by the above methods with other optional ingredients to form the dosage forms.

It is preferable that the food composition be a product in which a third-order function of food, that is, a physical condition adjustment function of food is emphasized when used as a food composition or as an ingredient of the food composition. Examples of products in which the third-order function of food is emphasized include health foods, foods with functional claims, foods with nutrient function, nutritional supplementary foods, supplements, and foods for specified health uses.

The pharmaceuticals, quasi-drugs, or food compositions consisting of the absorption enhancer according to the present embodiment, or the pharmaceuticals, quasi-drugs, or food compositions comprising the absorption enhancer may be for use in enhancing absorption of the cinnamic acid derivative. Further, labels may be attached to these products, the labels indicating, for example, that the product enhances absorption of the cinnamic acid derivative represented by formula (1) or (2), that the product enhances absorption of the active ingredient in propolis, that the product enhances absorption of propolis with high efficiency, that the product enhances absorption of propolis into the body, and that the product enhances the transfer of an active ingredient in propolis to blood.

### EXAMPLES

Hereinafter, the present invention will be more specifically described based on Examples. However, the present invention is not limited to the following Examples.

<Test Substance Administration Liquid>

The following administration liquids were prepared. 10% propylene glycol: a propylene glycol liquid having a concentration of 10% was prepared by diluting propylene glycol with distilled water.

Propolis solution: the required amount of propolis powder (Brazilian green propolis) was weighed and ground in a mortar, and the 10% propylene glycol was added to dissolve the powder to prepare a propolis solution with a concentration of 25 mg/mL as the propolis concentration.

Curcumin suspension: the required amount of curcumin powder (C. longa extract (ethyl acetate extract powder, curcumin content of 95% by mass)) was weighed and ground in a mortar, and then the 10% propylene glycol was added to suspend the powder to prepare a curcumin suspension with a curcumin concentration of 100 mg/mL.

<Tested Animal>

Eighteen 7-week-old male rats (Slc: Wistar/ST, SPF, Japan SLC, Inc.) were acclimated for 7 days. The rats were allowed to freely ingest the solid feed CRF-1 (Oriental Yeast Co., ltd.). The rats were fasted for 8 to 10 hours before the administration of the administration liquids. The rats were divided into three groups of six rats each by a stratified allocation method using EXSUS (Version 10.0.0, EP Croit Co., Ltd.) so that the weight of each group was equal.

<Administration>

In the propolis group, the propolis solution was administered immediately after the administration of 10% propylene glycol. In the propolis+curcumin A group, the propolis solution was administered immediately after the administration of the curcumin suspension. In the propolis+curcumin B group, the propolis solution was administered about 30 minutes after the administration of the curcumin suspension. The administration was carried out in a volume of 2 ml/kg rat body weight for each administration solution. In other words, 50 mg of propolis and 200 mg of curcumin were administered per 1 kg of rat body weight. The administration was carried out under no anesthesia by using a syringe (Terumo Corporation) and an oral feeding needle for rats (Fuchigami Kikai, Ltd).

<Blood Collection>

Blood collection of rats was carried out once 30 minutes before the administration of propolis solution (but before the administration of curcumin suspension in the group with the curcumin suspension administrated) and 0.5, 1, 1.5, 2, 3, 6, 9, and 24 hours after the administration of propolis solution. At each blood sampling time, the tail of rats was disinfected with 70% ethanol under no anesthesia, and then the tip of the tail was cut with a razor to collect about 150 μL of leaking blood by using a hematocrit tube (Fisher Scientific 22-362-566). Nine hours after administration of the propolis solution, feeding was resumed for all fasting animals.

The blood, which had been stored in ice after collection, was centrifuged (1600 ×g, 10 min, 4° C.) using a micro high-speed cooling centrifuge (model MX-100: Tomy Seiko Co., Ltd.) to collect plasma from the blood. The plasma was frozen and stored in a −80° C. freezer.

The concentrations of p-coumaric acid, artepillin C, drupanin, culifolin, capillartemisin A, 3,4-dihydroxy-5-prenyl-(E)-cinnamic acid, and 2,2-dimethylchromene-6-(E)-propenoic acid, kaempferide, 6-methoxy kaempferide, and dihydro kaempferide in the resulting plasma were quantified by LCMS.

For each ingredient, the maximum blood concentration (Cmax) and the area under the curve (AUC) were calculated from the resulting concentrations. AUC is an indicator of the total amount of each ingredient absorbed into the body. The results of the absorption of cinnamic acid derivatives in the propolis group and the propolis +curcumin A group are shown in Table 2. The ratio in the table indicates the ratio of the mean value of the propolis+curcumin A group to that of the propolis group. Statistics were carried out by t-test.

TABLE 2

| | | Cmax | | | AUC | | |
|---|---|---|---|---|---|---|---|
| | | Mean ± SD | P-value | Ratio | Mean ± SD | P-value | Ratio |
| p-Coumaric acid | Propolis | 343.72 ± 41.6 | 0.012 | 1.42 | 983.44 ± 372.02 | 0.681 | 1.07 |
| | Propolis + | 489.53 ± 98.09 | | | 1055.15 ± 73.61 | | |
| | curcumin A group | | | | | | |
| Artepillin C | Propolis | 77.7 ± 13.11 | 0.013 | 1.7 | 811.33 ± 191.31 | 0.047 | 1.39 |
| | Propolis + | 131.77 ± 38 | | | 1131.41 ± 250.48 | | |
| | curcumin A group | | | | | | |
| Drupanin | Propolis | 93.12 ± 19.93 | 0.0002 | 1.98 | 486.19 ± 34.29 | 0.001 | 1.41 |
| | Propolis + | 184.52 ± 30.33 | | | 685.09 ± 81.15 | | |
| | curcumin A group | | | | | | |
| Culifolin | Propolis | 68.98 ± 5.16 | 0.0001 | 1.34 | 1088.92 ± 68.35 | 0.002 | 1.26 |
| | Propolis + | 92.51 ± 6.27 | | | 1368.28 ± 131.94 | | |
| | curcumin A group | | | | | | |
| Capillartemisin A | Propolis | 10.39 ± 2.01 | 0.004 | 1.91 | 109.74 ± 12.25 | 0.012 | 1.83 |
| | Propolis + | 19.88 ± 5.39 | | | 201.32 ± 66.04 | | |
| | curcumin A group | | | | | | |
| 3,4-dihydroxy-5- | Propolis | 7.5 ± 7.5 | 0.047 | 1.53 | 51.02 ± 28.27 | 0.524 | 1.25 |
| prenyl-(E)-cinnamic | Propolis + | 11.49 ± 3.59 | | | 63.87 ± 33.12 | | |
| acid | curcumin A group | | | | | | |
| 2,2- | Propolis | 18.48 ± 4.02 | 0.059 | 1.56 | 84.5 ± 4.49 | 0.003 | 1.27 |
| dimethylchromene-6- | Propolis + | 28.86 ± 10.14 | | | 107.71 ± 12.21 | | |
| (E)-propenoic acid | curcumin A group | | | | | | |

For all cinnamic acid derivatives in Table 2, it was observed that at least either one of Cmax and AUC increased with a significant difference (p-value less than 0.05 or less than 0.01.) when curcumin and propolis were administrated in combination, compared to the administration of propolis alone. Also in the propolis+curcumin B group, in which a propolis liquid was administrated 30 minutes after the administration of a curcumin suspension, an increase in at least either one of Cmax and AUC was observed with a significant difference as in the A group, compared to the administration groups of the propolis alone. Regarding kaempferide, 6-methoxy kaempferide, and dihydro kaempferide, in contrast, no significant increase in absorption was observed in either of the propolis+curcumin A group and B group, when compared to the propolis groups.

What is claimed is:

1. A method for enhancing absorption of a cinnamic acid derivative, comprising:
administrating an effective amount of curcumin to a subject in need thereof, wherein the cinnamic acid derivative is at least one selected from the group consisting of a compound represented by formula (1): 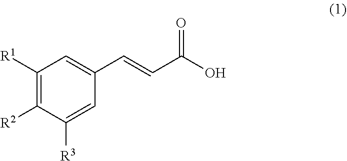
wherein R¹ represents a hydrogen atom, a hydroxyl group, a dimethylallyl group, 3-formyl-2-butenyl group, or (E)-3-methyl-4-hydroxy-2-butenyl group; R² represents a hydrogen atom, a hydroxyl group, or a dihydrocinnamoyloxy group; and R³ represents a hydrogen atom, a hydroxyl group, or a dimethylallyl group, or formula (2): 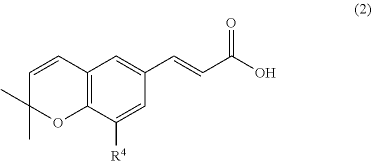
wherein R⁴ represents a hydrogen atom or a dimethylallyl group.

2. The method according to claim 1, wherein the cinnamic acid derivative is derived from propolis.

3. The method according to claim 1, wherein the cinnamic acid derivative is at least one selected from the group consisting of artepillin C, drupanin, p-coumaric acid, culifolin, capillartemisin A, 3,4-dihydroxy-5-prenyl-(E)-cinnamic acid, and 2,2-dimethylchromene-6-(E)-propenoic acid.
